(19) United States
(12) Patent Application Publication
(10) Pub. No.: US 2010/0247495 A1
(43) Pub. Date: Sep. 30, 2010

(54) Treatment of Muscular Dystrophy

(76) Inventors: Tom Ichim, San Diego, CA (US); Neil Riordan, Chandler, AZ (US)

Correspondence Address: BAUMGARTNER PATENT LAW, 3439 NE Sandy Blvd, Suite 285, PORTLAND, OR, 97232, US

(21) Appl. No.: 12/730,145

(22) Filed: Mar. 23, 2010

Related U.S. Application Data
(60) Provisional application No. 61/164,810, filed on Mar. 30, 2009.

(51) Int. Cl. A61K 35/14; A61K 35/50; A61K 35/48; A61P 21/00

(52) U.S. Cl. 424/93.7

(57) ABSTRACT

The present invention provides mesenchymal stem cells and mesenchymal-like cells useful for treatment of muscular dystrophies including Duchenne Muscular Dystrophy (DMD), as well as, Becker, limb girdle, congenital, facioscapulohumeral, myotonic, oculopharyngeal, distal, and Emery-Dreifuss dystrophies. Also provided are protocols for administration of cells for treatment of the above dystrophies and adjuvant protocols. Futhermore, the invention teaches methods of manipulating mesenchymal and mesenchymal-like cells in vitro and in vivo for augmentation of therapeutic effects. Particularly, use of endometrial regenerative cells, alone or in combination with mesenchymal stem cells is provided for treatment of DMD and Becker muscular dystrophy.

### CROSS-REFERENCE TO RELATED APPLICATIONS

This application claims priority to Provisional Application Ser. No. 61/164,810, filed Mar. 30, 2009 and entitled “Treatment of Muscular Dystrophy”, which is hereby expressly incorporated by reference in its entirety.

### FIELD OF THE INVENTION

The invention relates to the area of treatment of muscular disorders by cellular interventions. Particularly the invention is directed to the use of allogeneic cells stem/progenitor cells for treatment of patients with various musculopathies such as Duchenne Muscular Dystrophy (DMD), as well as, Becker, limb girdle, congenital, facioscapulohumeral, myotonic, oculopharyngeal, distal, and Emery-Dreifuss dystrophies. More specifically the invention is directed towards use of allogeneic mesenchymal and/or endometrial regenerative cells for inhibition of muscular damage, progression of muscular damage, and regeneration of muscle.

### BACKGROUND

Duchenne Muscular Dystrophy (DMD) is a lethal X-linked genetic disorder caused by a deficient dystrophin production. Mutations in the DMD gene, or duplications/deletions of its exons appears to be the underlying defect (1). Dystrophin is a critical component of the dystrophin glycoprotein complex (DGC), which is involved in stabilizing interactions between the sarcolemma, the cytoskeleton, and the extracellular matrix of skeletal and cardiac muscles (2). A consequence of the DGC inefficiency is the enhanced rate of myofibre death during muscle contraction. Although satellite cells compensate for muscle fibre loss in the early stages of disease (3), eventually these progenitors become exhausted as witnessed by shorter telomere length and inability to generate new muscle (4). Subsequently fibrous and fatty connective tissue overtaking the myofibres, in the process inflammatory cell infiltration, cytokine production and complement activation is observed (5, 6). At the clinical level, these changes culminate in progressive muscle wasting, with majority of patients being wheelchair-bound in their early teens. Patients succumb to cardiac/respiratory failure in their twenties, although rare cases of survival into the thirties has been reported (7).

With exception of corticosteroids, which have limited activity and carry numerous adverse effects (8), therapeutic interventions in DMD have had limited, if any success. Current areas of investigation include replacement gene therapy with dystrophin (9), induction of exon-skipping by antisense or siRNA to correct the open reading frame of mutated DMD genes (10), and transfer of myoblast or other putative progenitor cells (11-13).

Accordingly, in the art there have been no descriptions of significant improvement in muscle function in patients with DMD. The current invention provides cells useful for treatment of DMD and other dystrophies, as well as protocols, and combination approaches.

### DETAILED DESCRIPTION OF THE ILLUSTRATED EMBODIMENTS

The invention provides methods of treatment of muscular dystrophies including Duchenne Muscular Dystrophy (DMD), as well as, Becker, limb girdle, congenital, facioscapulohumeral, myotonic, oculopharyngeal, distal, and Emery-Dreifuss dystrophies through administration of allogeneic adherent stem cells such as mesenchymal stem cells. Particularly, the unexpected finding that mesenchymal stem cells obtained from placenta, placental body, Wharton's Jelly, and cord blood can be useful in clinical situations alone, or in combination with endometrial regenerative cells (ERC). Further disclosed is the use of ERC alone as a therapy for muscular dystrophies.

Muscle degeneration associated with DMD seems to be a multifactorial process in which numerous levels of intervention may be envisioned. Although induction of dystrophin expression is paramount to cure, factors such as inhibition of inflammation, suppression of ongoing fibrosis, and preserving cells for undergoing rapid apoptosis may all contribute to extending patient life-span. It is known that the inflammatory-associated transcription factor NF-kB is found upregulated in muscles of both animal models of DMD and clinical situations and that its inhibition results in therapeutic benefit (14). Further involved in the self-perpetuating inflammatory cascade is the renin-angiotensin system which increases the fibrotic cytokine TGF-b (15), and upregulation of TNF-alpha which is directly toxic to myocytes (16, 17). The active production of these inflammatory factors by infiltrating macrophages has been shown to play a large role in disease progression. M1 macrophages have been demonstrated to directly kill myocytes in vitro, whereas healing of muscles is associated with M2 macrophages, thus manipulation of this overall inflammatory state may be a potential area of intervention (18). However to date, inhibition of inflammation has been performed primarily through administration of steroids which have numerous adverse effects. Accordingly what is needed is an anti-inflammatory approach capable of concurrently stimulating muscle regeneration.

In one embodiment the invention provides methods of suppressing inflammatory conditions associated with muscle degeneration in muscular dystrophies such as DMD. Specifically, the invention teaches administration of allogeneic adherent cells, such as mesenchymal stem cells, or ERC, as anti-inflammatory cells. Said cells maybe collected, purified and expanded according to methods known in the art. For example, this has been described in several publications (Sun et al. In Vitro Proliferation and Differentiation of Human Mesenchymal Stem Cells Cultured in Autologous Plasma Derived from Bone Marrow. Tissue Eng Part A. 2008 March; 14 (3):391-400; Ball et al. Cotransplantation of ex vivo expanded mesenchymal stem cells accelerates lymphocyte recovery and may reduce the risk of graft failure in haploidentical hematopoietic stem-cell transplantation. Blood. 2007 Oct. 1; 110(7):2764-7; Schuleri et al. Mesenchymal stem cells for cardiac regenerative therapy. Handb Exp Pharmacol. 2007; (180):195-218).

In another embodiment, inhibition of inflammation maybe achieved through administration of endometrial regenerative cells, clinical production of which has previously been described by us and incorporated by reference (19).

Assessment of inhibition of inflammatory effects maybe performed so as to tailor patient dose. Particularly assessment of plasma inflammatory markers such as TNF or CRP maybe performed, or local detection of inflammation such as complement degradation products may be performed (6).

The increased fibrotic state of muscles in DMD is associated with upregulated expression of MMP inhibitors such as TIMP1 and 2 in patients (20). Modification of the MMP/TIMP ratio by administration of MMP overexpressing cells has yielded therapeutic benefit in the mdx model, which were associated with increased neovascularization (21). In fact, altered blood vessels were cited as a possible cause of DMD in historical literature (22). Administration of a cell type that can concurrently: a) differentiate into dystrophin expressing cells; b) possesses anti-inflammatory properties; c) stimulates remodeling/angiogenesis and thus reduces fibrosis; and d) inhibits premature satellite cell/myocyte apoptosis may be a therapeutically attractive strategy. ERC and various mesenchymal stem cells appear to fit this property but prior to the current disclosure have not been used or optimized for treatment of muscular dystrophy associated fibrosis. One embodiment of the invention is administration of mesenchymal or ERC cells either alone or after optimization for enhanced antifibrotic properties for treatment of muscular dystrophy.

Cells maybe administered intramuscularly in a patient along the major muscles, or maybe administered intravenously. Administration of cells maybe guided by therapeutic response.

### EXAMPLES

### Example 1

### Beckers Muscular Dystrophy

A 32 y.o. patient with Beckers Muscular Dystrophy was treated with multiple intramuscular injections of ERC cells which had been isolated and expanded from a donor under GTP conditions. The cells were taken from thaw, washed twice, and placed into syringes. Approximately 35 intramuscular injections of 3 million cells each of ERC were performed into multiple muscle groups. ERC were prepared as previously described (19, 23). Approximately 6 months after administration the patient was able to ambulate without the use of his cane and had gained significant strength in his trunk, legs, and arms.

### Example 2

### Duchenne Muscular Dystrophy

A 22 y.o. patient with a biopsy-confirmed case of Duchenne Muscular Dystrophy presented for treatment. The patient had been wheelchair-bound for approximately 12 years. He had severe limitation of mobility and muscle wasting. He was able to extend his hands 4 inches away from his body only. His maximum inspiratory pressure was 30 pre-treatment.

In August, 2008, approximately 90 million allogeneic (not-matched) menstrual-derived ERCs, prepared as above, were injected into his muscles groups over the 10 day period of treatment. The administration of a total of approximately 30 injections 3 million ERC each intramuscularly well tolerated.

At a 60-day follow up, the patient was able to blow up a balloon, which had not had the strength to do for 5 years prior. He was also able to hold his head erect, which was not possible before treatment. All of his major muscle groups increased in size, and his maximum inspiratory pressure was 40.

The patient was re-treated approximately 4 months after the initial treatment with approximately the same number of cells injected intramuscularly with ERCs and expanded mesenchymal cells derive from umbilical cord matrix. Approximately 60 days after the second treatment a muscle biopsy of the left quadriceps demonstrated dystrophin levels equivalent to normal controls and of normal size. At this time point the patient was able to resist forward and downward pressure on his head, lift 2 lb. weights overhead, and walk in a swimming pool with the aid of leg weights.

### REFERENCES

1. Muntoni, F., Torelli, S., and Ferlini, A. 2003. Dystrophin and mutations: one gene, several proteins, multiple phenotypes. Lancet Neurol 2:731-740.

2. Lapidos, K. A., Kakkar, R., and McNally, E. M. 2004. The dystrophin glycoprotein complex: signaling strength and integrity for the sarcolemma. Circ Res 94:1023-1031.

3. Miller, J. B., Schaefer, L., and Dominov, J. A. 1999. Seeking muscle stem cells. Curr Top Dev Biol 43:191-219.

4. Lund, T. C., Grange, R. W., and Lowe, D. A. 2007. Telomere shortening in diaphragm and tibialis anterior muscles of aged mdx mice. Muscle Nerve 36:387-390.

5. Cossu, G., and Mavilio, F. 2000. Myogenic stem cells for the therapy of primary myopathies: wishful thinking or therapeutic perspective? J Clin Invest 105:1669-1674.

6. Spuler, S., and Engel, A. G. 1998. Unexpected sarcolemmal complement membrane attack complex deposits on nonnecrotic muscle fibers in muscular dystrophies. Neurology 50:41-46.

7. Eagle, M., Baudouin, S. V., Chandler, C., Giddings, D. R., Bullock, R., and Bushby, K. 2002. Survival in Duchenne muscular dystrophy: improvements in life expectancy since 1967 and the impact of home nocturnal ventilation. Neuromuscul Disord 12:926-929.

8. Fenichel, G. M., Florence, J. M., Pestronk, A., Mendell, J. R., Moxley, R. T., 3rd, Griggs, R. C., Brooke, M. H., Miller, J. P., Robison, J., King, W., et al. 1991. Long-term benefit from prednisone therapy in Duchenne muscular dystrophy. Neurology 41:1874-1877.

9. Romero, N. B., Braun, S., Benveniste, O., Leturcq, F., Hogrel, J. Y., Morris, G. E., Barois, A., Eymard, B., Payan, C., Ortega, V., et al. 2004. Phase I study of dystrophin plasmid-based gene therapy in Duchenne/Becker muscular dystrophy. Hum Gene Ther 15:1065-1076.

10. van Deutekom, J. C., Janson, A. A., Ginjaar, I. B., Frankhuizen, W. S., Aartsma-Rus, A., Bremmer-Bout, M., den Dunnen, J. T., Koop, K., van der Kooi, A. J., Goemans, N. M., et al. 2007. Local dystrophin restoration with antisense oligonucleotide PRO051. N Engl J Med 357:2677-2686.

11. Torrente, Y., Belicchi, M., Marchesi, C., Dantona, G., Cogiamanian, F., Pisati, F., Gavina, M., Giordano, R., Tonlorenzi, R., Fagiolari, G., et al. 2007. Autologous transplantation of muscle-derived CD133+stem cells in Duchenne muscle patients. Cell Transplant 16:563-577.

12. Law, P. K., Goodwin, T. G., Fang, Q., Duggirala, V., Larkin, C., Florendo, J. A., Kirby, D. S., Deering, M. B., Li, H. J., Chen, M., et al. 1992. Feasibility, safety, and efficacy of myoblast transfer therapy on Duchenne muscular dystrophy boys. Cell Transplant 1:235-244.

13. Mendell, J. R., Kissel, J. T., Amato, A. A., King, W., Signore, L., Prior, T. W., Sahenk, Z., Benson, S., McAndrew, P. E., Rice, R., et al. 1995. Myoblast transfer in the treatment of Duchenne's muscular dystrophy. N Engl J Med 333:832-838.

14. Acharyya, S., Villalta, S. A., Bakkar, N., Bupha-Intr, T., Janssen, P. M., Carathers, M., Li, Z. W., Beg, A. A., Ghosh, S., Sahenk, Z., et al. 2007. Interplay of IKK/NF-kappaB signaling in macrophages and myofibers promotes muscle degeneration in Duchenne muscular dystrophy. J Clin Invest 117:889-901.

15. Sun, G., Haginoya, K., Dai, H., Chiba, Y., Uematsu, M., Hino-Fukuyo, N.,

Onuma, A., Iinuma, K., and Tsuchiya, S. 2009. Intramuscular renin-angiotensin system is activated in human muscular dystrophy. J Neurol Sci.

16. Radley, H. G., Davies, M. J., and Grounds, M. D. 2008. Reduced muscle necrosis and long-term benefits in dystrophic mdx mice after cVlq (blockade of TNF) treatment. Neuromuscul Disord 18:227-238.

17. Hodgetts, S., Radley, H., Davies, M., and Grounds, M.D. 2006. Reduced necrosis of dystrophic muscle by depletion of host neutrophils, or blocking TNFalpha function with Etanercept in mdx mice. Neuromuscul Disord 16:591-602.

18. Villalta, S. A., Nguyen, H. X., Deng, B., Gotoh, T., and Tidball, J. G. 2009. Shifts in macrophage phenotypes and macrophage competition for arginine metabolism affect the severity of muscle pathology in muscular dystrophy. Hum Mol Genet 18:482-496.

19. Zhong, Z., Patel, A. N., Ichim, T. E., Riordan, N. H., Wang, H., Min, W. P., Woods, E. J., Reid, M., Mansilla, E., Marin, G. H., et al. 2009. Feasibility investigation of allogeneic endometrial regenerative cells. J Transl Med 7:15.

20. von Moers, A., Zwirner, A., Reinhold, A., Bruckmann, O., van Landeghem, F., Stoltenburg-Didinger, G., Schuppan, D., Herbst, H., and Schuelke, M. 2005. Increased mRNA expression of tissue inhibitors of metalloproteinase-1 and -2 in Duchenne muscular dystrophy. Acta Neuropathol 109:285-293.

21. Gargioli, C., Coletta, M., De Grandis, F., Cannata, S. M., and Cossu, G. 2008. P1GF-MMP-9-expressing cells restore microcirculation and efficacy of cell therapy in aged dystrophic muscle. Nat Med 14:973-978.

22. Musch, B. C., Papapetropoulos, T. A., McQueen, D. A., Hudgson, P., and Weightman, D. 1975. A comparison of the structure of small blood vessels in normal, denervated and dystrophic human muscle. J Neurol Sci 26:221-234.

23. Ichim, T. E., Solano, F., Brenes, R., Glenn, E., Chang, J., Chan, K., and Riordan, N. H. 2008. Placental mesenchymal and cord blood stem cell therapy for dilated cardiomyopathy. Reprod Biomed Online 16:898-905.

1. A cellular composition useful for treatment of a muscular dystrophy, said composition comprising an adherent population of cells derived from a group comprising of: the placental body, cord blood, Wharton's Jelly, menstrual blood, endometrium, and amniotic fluid, administered at a concentration, frequency, and location sufficient to induce improvement in muscle function or inhibit deterioration of muscle function in a patient suffering from a muscular dystrophy.

2. The cellular composition of claim 1, wherein said muscular dystrophy is selected from a group of muscular dystrophies comprising of Duchenne, Becker, limb girdle, congenital, facioscapulohumeral, myotonic, oculopharyngeal, distal, and Emery-Dreifuss dystrophies.

3. The cellular composition of claim 1, wherein said adherent cell population is a mesenchymal stem cell.

4. The mesenchymal stem cell of claim 3, wherein said cell expresses >90% CD90 and CD105 and <5% CD14, CD34, and CD45.

5. The mesenchymal stem cell of claim 3, wherein said cell expresses one or more markers selected from the group consisting of: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

6. The method of claim 1, wherein said adherent cell is allogeneic.

7. The method of claim 1, wherein said adherent cell is matched by mixed lymphocyte reaction matching.

8. The method of claim 1, wherein said adherent cell is an Endometrial Regenerative Cell (ERC).

9. The method of claim 8, wherein said ERC is a human pluripotent stem cell that expresses a marker selected from CD29, CD41a, CD44, CD90, and CD105, and having an ability to proliferate at a rate of 0.5-1.5 doublings per 24 hours in a growth medium.

10. The ERC of claim 8, wherein said cell further expresses a marker selected from NeuN, CD9, CD62, CD59, Actin, GFAP, NSE, Nestin, CD73, SSEA-4, hTERT, Oct-4, and tubulin.

11. ERC of claim 8, wherein said cell further expresses a marker selected from hTERT and Oct-4, but does not express a STRO-1 marker, and has an ability to undergo cell division in less than 24 hours in a growth medium.

12. The ERC of claim 8, wherein said cell further expresses a STRO-1 marker, and has an ability to proliferate at a rate of 0.5-0.9 doublings per 24 hours in a growth medium.

13. The ERC of claim 8, wherein said cell produces matrix metalloprotease 3 (MMP3), matrix metalloprotease 10 (MMP10), GM-CSF, PDGF-BB or angiogenic factor ANG-2.

14. The ERC of claim 8, wherein said cell is derived or originates from endometrium, endometrial stroma, endometrial membrane, or menstrual blood.

15. The method of claim 1, wherein said cell is administered intramuscularly, intravenously, or in a combination.

16. A cellular composition useful for treatment of muscular fibrosis associated with muscular dystrophy, said composition comprising an adherent population of cells derived from a group comprising of: the placental body, cord blood, Wharton's Jelly, menstrual blood, endometrium, and amniotic fluid, administered at a concentration, frequency, and location sufficient to induce improvement in muscle function or inhibit deterioration of muscle function in a patient suffering from a muscular dystrophy.

17. The cellular composition of claim 16, wherein said muscular dystrophy is selected from a group of muscular dystrophies comprising of Duchenne, Becker, limb girdle, congenital, facioscapulohumeral, myotonic, oculopharyngeal, distal, and Emery-Dreifuss dystrophies.

18. The cellular composition of claim 16, wherein said adherent cell population is a mesenchymal stem cell.

19. The mesenchymal stem cell of claim 18, wherein said cell expresses >90% CD90 and CD105 and <5% CD14, CD34, and CD45.

20. The mesenchymal stem cell of claim 18, wherein said cell expresses one or more markers selected from the group consisting of: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1.

21. The method of claim 16, wherein said adherent cell is allogeneic.

22. The method of claim 16, wherein said adherent cell is matched by mixed lymphocyte reaction matching.

23. The method of claim 16, wherein said adherent cell is an Endometrial Regenerative Cell (ERC).

24. The method of claim 23, wherein said ERC is a human pluripotent stem cell that expresses a marker selected from CD29, CD41a, CD44, CD90, and CD105, and having an ability to proliferate at a rate of 0.5-1.5 doublings per 24 hours in a growth medium.

25. The ERC of claim 23, wherein said cell further expresses a marker selected from NeuN, CD9, CD62, CD59, Actin, GFAP, NSE, Nestin, CD73, SSEA-4, hTERT, Oct-4, and tubulin.

26. ERC of claim 23, wherein said cell further expresses a marker selected from hTERT and Oct-4, but does not express a STRO-1 marker, and has an ability to undergo cell division in less than 24 hours in a growth medium.

27. The ERC of claim 23, wherein said cell further expresses a STRO-1 marker, and has an ability to proliferate at a rate of 0.5-0.9 doublings per 24 hours in a growth medium.

28. The ERC of claim 23, wherein said cell produces matrix metalloprotease 3 (MMP3), matrix metalloprotease 10 (MMP10), GM-CSF, PDGF-BB or angiogenic factor ANG-2.

29. The ERC of claim 23, wherein said cell is derived or originates from endometrium, endometrial stroma, endometrial membrane, or menstrual blood.

30. The method of claim 16, wherein said cell is administered intramuscularly, intravenously, or in a combination.
